SEQUENCE LISTING (5 sequences)

SEQ ID NO: 1 - DNA, 22 bp, Artificial Sequence
  Primer tDT-1
ttgtacagctcgtccatgccgt

SEQ ID NO: 2 - DNA, 20 bp, Artificial Sequence
  Primer tDT-2
aagaagaccatgggctggga

SEQ ID NO: 3 - DNA, 20 bp, Artificial Sequence
  Primer nosTxxxf01
gaatttccccgatcgttcaa

SEQ ID NO: 4 - DNA, 21 bp, Artificial Sequence
  Primer nosTxxxr03
ccggcaacaggattcaatctt

SEQ ID NO: 5 - DNA, 15 bp, Artificial Sequence
  Primer nosTxxxMGB combined with FAM
catttggcaataaag

(19) United States
(12) Patent Application Publication
(10) Pub. No.: US 2016/0312235 A1
(43) Pub. Date: Oct. 27, 2016

(54) SELECTION MARKER-FREE RHIZOBIACEAE-MEDIATED METHOD FOR PRODUCING A TRANSGENIC PLANT OF THE TRITICUM GENUS

(71) Applicant: KWS SAAT SE, Einbeck (DE)

(72) Inventor: Klaus Schmidt, Lahstedt (DE)

(73) Assignee: KWS SAAT SE, Einbeck (DE)

(21) Appl. No.: 15/104,790

(22) PCT Filed: Dec. 13, 2014
(86) PCT No.: PCT/DE2014/000639

§ 371 Date: Jun. 15, 2016

(30) Foreign Application Priority Data
Dec. 15, 2013 (DE) 10 2013 020 605.7

(51) Int. Cl. C12N 15/82; C12Q 1/68

(52) U.S. Cl. CPC C12N 15/8205; C12Q 1/6895; C12N 15/8274; C12Q 2600/158

(57) ABSTRACT

The present invention provides an improved method for producing a transgenic plant of the Triticum genus comprising the steps (a) Rhizobiaceae-mediated transformation of at least one cell of a plant of the Triticum genus with a genetic component and (b) regeneration of a transgenic plant of the Triticum genus from a transformed cell, wherein from step (a) to step (b) there is no selection of a transformed cell based on a property mediated by the genetic component or a portion thereof.

### FIELD OF THE INVENTION

The present invention relates to the field of biotechnology and includes an improved method for producing a transgenic plant of the Triticum genus by using bacterial cells from the Rhizobiaceae family, in particular the agrobacterium genus as well as transgenic plants or parts thereof which were produced by the improved method.

### BACKGROUND OF THE INVENTION

Products of the plants of the Triticum genus, such as wheat (Triticum aestivum), are one of the most important raw materials and play an important role as basic nutrients throughout most of the world. Nevertheless, in the last 50 years, the progress achieved with wheat through conventional cultivation has lagged significantly behind that of other types of crops, such as maize, sugar beet or rapeseed with regard to a wide variety of aspects, such as yield. The development of transgenic plants of the Triticum genus is one possibility of making up for this lack of progress to at least some extent. However, production of transgenic plants of the Triticum genus by Rhizobiaceae (e.g., Agrobacterium tumefaciens)-mediated transformation has always been considered extremely difficult. Efficiencies of only 1-3% transgenic lines per isolated starting explant are usually achieved here in the case of wheat, for example. In individual cases, there have been reports in the literature of transformation protocols with efficiencies of up to 10% (Hensel et al., 2009; Shrawat and Lorz, 2006), but these efficiencies often cannot be achieved in practice. Known protocols include almost exclusively the use of marker genes for selection (selection markers) in a co-transformation. The selection marker is usually coupled to the gene of interest (goi) to be transformed. The marker gene is usually either an antibiotic resistance gene or a herbicide resistance gene, which imparts a survival advantage to the transformed cells under certain in vitro conditions during the regeneration phase. Marker genes thus offer a way to differentiate transgenic plants from non-transgenic plants. Ultimately the use of selection with marker genes permits a more efficient transformation and/or makes the transformation possible for the first time.

Since the selection marker is needed in the transgenic plant only during the in vitro phase, it no longer fulfills any function later in the plant and is therefore superfluous at that point. However, since the number of available selection markers is limited, the presence of the selection marker that is no longer needed complicates and makes more difficult a subsequent supertransformation of the plant that is already transgenic with a second gene of interest (goi). Stacking of multiple genes by means of sequential transformation is thus possible only to a limited extent and is also limited by the number of different selection markers that are available for the respective plant species.

In addition, the use of antibiotic resistance genes as selection markers in transgenic plants is being criticized in the public in particular so that basically only transgenic plants without selection marker are acceptable in regulatory approval and in commercialization. However, removing the selection marker is associated with a great effort in terms of labor, cost and time.

Various methods and aids are available today to those skilled in the art for removing a selection marker from the genome of a transgenic line. First, highly specific nucleases (e.g., zinc finger nucleases) can be used. By crossing with a nuclease-expressing line, such nucleases must be introduced into the genome of the transgenic plants containing the selection marker to be removed. After successful elimination of the selection marker, it is still necessary to remove the nuclease from the genome of the transgenic plant, which is accomplished by means of meiotic segregation. Therefore, at least two additional generations are needed for identification of selection marker-free plants. The use of specific recombinases (e.g., Cre-recombinase) can be regarded as one variant of this method, but these always result in a persistence of the recombination sites in the transgenic plant. This is problematical from a regulatory standpoint because this also involves unnecessary, i.e., superfluous, sequence motifs within the transgenic plant.

Furthermore, the plants can be transformed with two T-DNAs, where one T-DNA carries the gene of interest (goi) and the other T-DNA carries the selection marker. In approximately 30% to 50% of the resulting transgenic plants, the T-DNAs are then integrated into one cell but at different locations in the genome. Segregation of the selection marker and the gene of interest (goi) in the subsequent generation is therefore possible by means of meiosis. However, selection marker-free plants cannot be identified until the first filial generation of the starting transformants. However, separation of selection markers and the gene of interest (goi) by segregation is highly inefficient due to the frequent co-integration of the two transformed T-DNAs into genomic regions that are close together, so that a large number of the starting transformants must be created in order to be able to identify a sufficient number of transgenic selection marker-free lines.

Production of transgenic plants without the use of a selection step during the transformation process was long considered to be impossible (Potrykus et al, 1998; Erikson et al., 2005; Joersbo et al., 2001). In their review article in the year 2006, Shrawat and Lorz describe various possibilities for producing selection marker-free cereal crop plants, but all the methods are based on the use of one of the strategies described above, i.e., either performing co-transformations (the gene of interest and the selection marker are then on two separate T-DNAs) with subsequent segregation of the selection marker and the gene of interest (goi) by meiosis or subsequent removal of the selection marker by means of specific recombinases. They do not describe the use of a selection marker-free transformation.

In a review article published recently by Tuteja et al. (2012), numerous methods of creating marker gene-free plants are also described, but again in this article, the possibilities of co-transformation and/or the subsequent selection marker removal, which are described by Shrawat and Lorz (2006), are mentioned only once. There is no mention of transformation without a selection marker implants of the Triticum genus by means of Rhizobiaceae bacteria such as Agrobacterium tumefaciens. Transformation of plants by means of agrobacterium without the presence and use of a selection marker has been described for a few other plant species including potato (De Vetten et al., 2003; Ahmad et al., 2008), tobacco (Li et al., 2009), orange (Ballester et al., 2010) and alfalfa (Ferradini et al, 2011).

Today there are the following unwanted phenomena that can occur if selection with a marker gene is omitted:

The transformed explant usually passes through several selection steps in the callus phase. During this selection phase, transgenic cells accumulate in the callus carrying the corresponding resistance gene, i.e., being transgenic, due to the presence of an antibiotic or a herbicide. Non-transgenic cells are inhibited in their growth and die off, which greatly increases the probability that mainly transgenic shoots will regenerate from the selected callus. Faize et al. (2010) have thus shown that, during the process of transformation of apricot, the amount of transgenic tissue in apricot shoots can be increased by repeated subculture on a selective medium, and thus a chimeric character of the shoot can be reduced or eliminated by using selection. If the selection steps are omitted, there is obviously the risk that the non-transgenic shoots will be superior to those from transgenic cells during regeneration. It is assumed that the transformed cells have a vitality disadvantage in comparison with non-transformed cells due to the agrobacterium infection. Thus in a selection marker-free transformation, there is an increase in the probability that predominantly non-transgenic shoots will regenerate. Consequently there is a significant decline in the transformation efficiency in comparison with a transformation with selection. This has been investigated very well in the case of selection marker-free potato transformation, in which efficiencies of 1-4% have been described (De Vette et al., 2003), whereas efficiencies of approximately 30% can be obtained in transformation with a selection marker (Chang and Chan, 1991).

Furthermore, it is regularly observed that in the absence of a marker gene-based selection, shoots that consists of both transgenic and non-transgenic tissue (chimeric shoots) will regenerate. Different forms of the chimeric character may be present. If a periclinal chimera should be present, it may happen that the L2 cell layer required for the development of the gametes in the meristems of the plants is not transgenic. Thus only non-transgenic gametes are formed in this plant and the transgene introduced into the plant will not be propagated to the next generation. Such chimeric transgenic plants are then lost in the case of plants to be reproduced generatively. In sectoral chimeric plants some regions of the plants are transgenic while other regions are not transgenic. Only non-transgenic gametes are formed in the non-transgenic regions/portions of the plant. The amount of non-transgenic gametes is definitely increased by this so that an increased amount of non-transgenic progeny can be detected in the subsequent generation. The split ratios in the filial generation then do not correspond to Mendel's laws. By using marker gene-based selection, the development of chimeric shoots is usually suppressed or the amount of transgenic tissue in a sectoral chimera is so high due to the selection pressure that there are very little or no negative effects of the chimeric character of the regenerated transgenic plant, in particular a heredity that does not conform to Mendel's laws.

For monocotyledonous crop plants, there are only a few applicable methods known in the state of the art for transformation and production of marker gene-free plants. In particular a successful selection marker-free production of transgender wheat plants has been described only by Liu et al., 2011. However, the yield achieved by this method is extremely low at only 0.28%, which is why the method they described is not suitable for routine use. Furthermore, the authors use micro-projectile bombardment for the transformation, but not Rhizobiaceae bacteria such as Agrobacterium tumefaciens.

WO 2008/028121 describes the creation of selection marker-free maize plants, which can be generated without the use of selection. These authors even propose also applying the method they disclose to other Poaceae, such as wheat, but the examples they describe are limited exclusively to the creation of transgenic maize plants. Furthermore, these authors state that the maize plants created should preferably not be chimeric but they do not provide any experimental data on the transmission of the transgene to the next generation so that the possibility cannot be ruled out that most of the transgenic maize lines created are chimeric. EP 2 274 973 also describes the creation of transgenic monocotyledonous plants, in particular maize and rice plants by means of agrobacterium-mediated transformation in which no selection step is used. It is shown clearly that for maize, a not insignificant number of chimeric plants are formed which must be identified and sorted out in a complex procedure. The amount of starting chimeric transformants was >50% of the transgenic shoots obtained. Only <20% of the transgenic plants generated were not all chimeric (uniform). Thus the number of transformants with a chimeric character is expected to be many times greater than is the case in transformation with corresponding selection steps. Thus, for example, Coussens et al. (2012) have shown that in generation of transgenic maize plants using the selection marker bar, an amount of only approximately 5% of the plants created is chimeric, i.e., 95% of the plants created are not chimeric and therefore the transgene is transmitted to the next generation in accordance with Mendel's laws. In addition, in EP 2 274 973, the authors describe transformation of rice without using a selection marker but they do not perform any analyses that would show how great the amount of chimeric plants in the population of generated selection marker-free plants was. It is interesting in this context that chimeric plants also occur in the transformation of rice using selection pressure (Hiei et al., 1994). It can therefore also be anticipated here that the amount of chimeric plants is definitely elevated in the rice in the selection marker-free transformation. The authors of EP 2 274 973 also propose using the production process disclosed there for creation of transgenic wheat but they do not provide any experimental data about which efficiencies and chimeric trends are to be expected with wheat. Although wheat, like maize and rice, are among the monocotyledonous plants, those skilled in the art are aware of the fact that cells of this crop plant species may exhibit great differences in behavior in the process of transformation and regeneration, which is why one must question the conclusion that the results of transformation of other monocotyledonous plants can be readily applied to wheat plants. Thus, Hensel et al., 2009, for example, also point out such differences in a comparison of the transformation of barley, maize, triticale and wheat. EP 2 460 402 A1 discloses a particularly efficient method of transforming wheat cells by means of Agrobacterium tumefaciens, which should permit yields of 70% or more transgenic lines per isolated starting explant in regeneration. However, the transformation protocol used here always includes the use of the selection marker hygromycin phosphotransferase (hpt) or phosphinothricin acetyltransferase (PAT/bar). To be sure, these authors do state that selection is not absolutely necessary for generation of transgenic wheat plants, but they do not provide any experimental proof of this statement.

### SUMMARY OF THE INVENTION

The present invention was developed against the background of the state of the art described above wherein the object of the present invention is to provide a Rhizobiaceae-mediated method for producing a transgenic plant of the Triticum genus, which does not require a marker gene-based selection and minimizes the unwanted effects described above or exhibits those effects only to a limited extent. In addition the object of the present invention is a method for producing a transgenic plant of the Triticum genus, which is superior to previous methods from both an economic standpoint and a regulatory standpoint.

These objects are achieved according to the invention by a method for producing a transgenic plant of the Triticum genus comprising the steps (a) transforming at least one cell of a plant of the Triticum genus with a genetic component by co-culturing cells of an explant of the plant of the Triticum genus with at least one bacterial cell from the Rhizobiaceae family comprising the genetic component and (b) regenerating a transgenic plant of the Triticum genus from at least one transformed cell from (a), wherein no selection of a transformed cell from (a) based on a property mediated by the genetic component or a portion thereof takes place from step (a) to step (b).

A bacterial cell from the Rhizobiaceae family is preferably a bacterial cell of the Agrobacterium genus and especially preferably a bacterial cell of the Agrobacterium tumefaciens species (Broothaerts et al., 2005). The bacterial cell preferably includes the genetic component on a vector, in particular on a binary vector, a super binary vector or a vector of a co-integrated vector system.

The genetic component is preferably a nucleic acid molecule, in particular a DNA molecule or a recombinant DNA and comprises at least the gene of interest. In addition, the genetic component may also have a regulatory sequence, an intron, a recognition sequence for an RNA molecule, a DNA molecule or a protein or a 5′- or 3′-UTR (untranslated region).

In a method according to the present invention, the transformation in step (a) can be carried out under conditions which allow successful infection of at least one cell of an explant of the plant of the Triticum genus with a bacterial cell from the Rhizobiaceae family. Those skilled in the art are familiar with such transformation conditions from the state of the art (Cheng et al., 1997). The explant used in step (a) is preferably an embryonal tissue, in particular radicula, embryoaxis, scutellum or nucleus or a portion thereof and represents a portion of an immature embryo or a mature gamete (EP 0 672 752 B1). However, other suitable tissues are also known that can be used successfully for transformation of plants of the Triticum genus such as wheat (Shrawat and Lorz, 2006).

In addition, regeneration of a transgenic plant of the Triticum genus from at least one transformed cell from (a) in step (b) also means regeneration of a plant from the transformed cell derived from at least one transformed cell from (a) by cell division, for example, as a result of formation of a callus, which is restructured into somatic embryos in order to then lead to shoot regeneration. Various techniques for regeneration of a plant of the Triticum genus are familiar to those skilled in the art from the state of the art. Regeneration may take place, for example, from immature embryos (Vasil et al., 1993). Another possibility of regeneration is derived from anthers or microspores (example: Maluszynski et al., 2003). Furthermore, wheat plants have also been regenerated from flower tissue (Amoah et al., 2001) and from the callus of immature embryos (Wang et al., 2009).

In the method according to the invention, from step (a) to step (b), there is no selection of a transformed cell from (a) based on a property mediated by the genetic component or a part thereof. A transformed cell from (a) here may also denote a transformed cell derived by cell division from at least one transformed cell from (a). There is preferably no selection based on a property mediated by the genetic component or a part thereof, and no selection based on a herbicide or antibiotic resistance.

Herbicide resistance can be achieved, for example, by expression of phosphinothricin acetyltransferase from Streptomyces hygroscopicus or Streptomyces viridochromogenes which mediates a resistance to the herbicide phosphinothricin, i.e., bialaphos (De Block et al., 1987). Another herbicide resistance namely resistance to the active ingredient glyphosate can be achieved by overexpression of 5-enolpyruvylshikimate-3-phosphate synthase. An enzyme that is insensitive to glyphosate is usually used for this purpose (Comai et al., 1983).

Furthermore, resistance to the herbicide classes of sulfonylureas, sulfonylaminocarbonyl-triazolinones, imidazolinones, triazolopyrimidines and pyrimidinyl(thio)benzoates can be achieved by expression of a mutagenized form of the enzyme acetolactate synthase (ALS). Different mutations lead to a resistance to the different herbicides. An overview of the herbicide resistances generally used can be found in Tuteja et al. (2012), Kraus (2010) or Shrawat and Lorz (2006).

Antibiotic resistance can be achieved by expression of bacterial genes, which inactivate the antibiotic used by transfer of a phosphate or acetyl group. Examples of this include neomycin phosphotransferase (npt), which mediates a resistance to antibiotics of the aminoglycoside class (e.g., kanamycin, paromomycin, geneticin). Hygromycin phosphotransferase which imparts a resistance to the antibiotic hygromycin B, for example, is used as another commonly used antibiotic resistance. An overview of antibiotic resistance that can be used in plant transformation can be found in Tuteja et al. (2012), Kraus (2010) or Shrawat and Lorz (2006).

However, in addition to antibiotic and herbicide resistance, other selection markers which permit differentiation between transgenic and non-transgenic cells may also be used. Examples include stimulation of production of anthocyans or other plant pigments by expression of certain transcription factors (Kortstee et al., 2011), expression of fluorescent proteins (Mussmann et al., 2011) or expression of auxotrophic markers such as phosphomannose isomerase (PMI), expression of which permits the growth of transgenic cells on mannose as the sole carbohydrate source, although non-transgenic cells are unable to use this carbon source (Reed et al., 2001).

Those skilled in the art are aware that, in addition to transgenic plants, also non-transgenic or chimeric plants can regenerate in step (b) because of the lack of selection pressure on the transformed cells as well as the non-transformed cells from step (a) to step (b) of the method according to the invention. The low yield of usable transgenic plants (non-chimeric) has for a long time stood in the way of an economically viable use of a marker gene-free method for producing a transgenic plant. As a rule, production of a transgenic plant with selection, based on a marker gene and subsequent removal of the selection marker, was still the method of choice for creating transgenic selection marker-free plants, although this was associated with enormous expenditures in terms of labor, costs and time. To increase the efficiency of creation of transgenic monocotyledonous plants, those skilled in the art are in agreement that this can be accomplished exclusively through the fact that the infection rate must already be increased significantly at the time of co-culturing of the cells of the explant with the Agrobacterium. This should then lead to an increased transformation rate, i.e., the presence of more transformed cells in the explant, from which then more transgenic plants should be regenerated. Various approaches for such enhanced transformation efficiency are known from the state of the art (US 2011/0030101 A1 ). These approaches have also been used successfully in methods for marker gene-free production of maize and rice. Nevertheless, even today, the marker gene-free methods of producing transgenic maize and rice plants still remain behind the methods with marker gene-based selection, so that the production of transgenic maize and rice plants still takes place mainly with the use of marker gene-based selection. This can also be attributed to a substantial extent to the persistent problems of increased generation of chimeric plants when omitting a selection marker and the subsequent need for identification and sorting of these plants. The amount of chimeric plants in the absence of marker gene-based selection is usually much greater in comparison with the amount obtained by using a marker gene.

The method according to the invention has described for the first time the production of a transgenic plant of the Triticum genus using a Rhizobiaceae-mediated transformation, in which there is no selection of a transformed cell based on a property mediated by the genetic component or a part thereof introduced during the transformation. Contrary to expectations, the method according to the present invention has yielded a surprisingly high transformation efficiency, which was much higher than the transformation efficiencies known from the state of the art for marker gene-free production processes of transgenic plants of the Triticum genus without the use of bacteria of the Rhizobiaceae family such as Agrobacterium tumefaciens. This method preferably has a transformation efficiency of at least 5%, 6%, 7%, 8%, 9% or 10%, especially preferably at least 11%, 12%, 13%, 14%,15%, 16%, 17%, 18%, 19%, 20% or most especially preferably at least 21%, 22%, 23%, 24%, 25%, 26%, 27%, 28%, 29%, 30%, 31%, 32%, 33%, 34%, 35%, 36%, 37%, 38%, 39%, 40% or more than 40%.

In a preferred embodiment of the method according to the invention, the transformation efficiency is comparable to the transformation efficiency of a corresponding comparative method, which differs in that it includes selection of a transformed cell based on a property mediated by the genetic component or a portion thereof, i.e., based on at least one selection marker. In addition the transformation efficiency of the method according to the invention may amount to at least 95%, at least 90%, at least 85%, at least 80%, at least 75%, at least 70%, at least 65%, at least 60%, at least 55%, at least 50%, at least 45%, at least 40%, at least 35%, at least 30% or at least 25% of the transformation efficiency of an equivalent method in which a selection of a transformed cell takes place based on a property mediated by the genetic component or a portion thereof, i.e., based on at least one selection marker. Because of the great effort involved, which is associated with the subsequent removal of the selection marker from stable transgenic plants, a skilled person will also regard the method according to the invention as advantageous and superior to the state of the art if such a transformation efficiency is achieved in the method according to the invention. Furthermore, such a high transformation efficiency should be surprising to those skilled in the art because they would expect a much lower transformation efficiency, based on experience with methods of marker gene-free production of transgenic maize and rice plants, for example.

In another preferred embodiment of the method according to the invention, the method described above is characterized in that the transformation efficiency is increased by treatment to increase the transformation efficiency. Treatment to increase the transformation efficiency may achieve a transformation efficiency of at least 5%, 6%, 7%, 8%, 9% or 10%, preferably at least 11%, 12%, 13%, 14%,15%, 16%, 17%, 18%, 19%, 20% or especially preferably at least 21%, 22%, 23%, 24%, 25%, 26%, 27%, 28%, 29%, 30%, 31%, 32%, 33%, 34%, 35%, 36%, 37%, 38%, 39%, 40% or more than 40%. Various treatment to increase a transformation efficiency and methods to produce a transgenic plant, in particular a transgenic monocotyledonous plant have been described in the prior art. The treatment to increase the transformation efficiency may include at least a treatment selected from:
i. physical and/or chemical damage to the tissue or a portion thereof during co-culturing or after co-culturing (EP 2 460 402),
ii. centrifugation before co-culturing, during co-culturing or after co-culturing (Hiei et al., 2006, WO 2002/012520),
iii. addition of silver nitrate and/or copper sulfate to the co-culturing medium (Zhao et al., 2002; Ishida et al., 2003; WO 2005/107152),
iv. thermal treatment of the explant before or during co-culturing (WO 1998/054961),
v. pressure treatment before co-culturing or during co-culturing or after co-culturing (WO 2005/017169),
vi. inoculation of Agrobacterium in the presence of a powder (WO 2007/069643) and
vii. addition of cysteine to the co-culturing medium (Frame et al., 2002).

In addition, other treatments to increase transformation efficiency are known from the state of the art and can be used in the method according to the present invention. Furthermore, a treatment to increase transformation efficiency may also comprise a combination of known treatment to increase transformation efficiency.

In another preferred embodiment of the method according to the invention, the method described above is characterized either by the fact that regeneration of a transgenic plant of the Triticum genus in step (b) brings forth non-chimeric transgenic plants with an incidence of at least 15%, at least 16%, at least 17%, at least 18%, at least 19%, at least 20%, at least 22%, at least 24%, at least 26%, at least 28%, at least 30%, at least 32%, at least 34%, at least 36%, at least 38% or at least 40%, preferably at least 45%, at least 50%, at least 55%, at least 60%, at least 65% or at least 70%, especially preferably at least 75%, at least 80%, at least 85% or at least 90%, or is characterized in that the regeneration of a transgenic plant of the Triticum genus in step (b) brings forth trimeric transgenic plants, preferably with an incidence of less than 70%, 65%, 60%, 55%, 50%, 45%, 40%, 35%, 30%, 28%, 26%, 24%, 22%, 20%, 18%, 16%, 15%, 14%, 13%, 12%, 11%, 10%, 9%, 8%, 7%, 6% or 5%.

In a particularly preferred embodiment of the method according to the invention, the amount of non-chimeric transgenic plants or the Triticum genus from step (b) is comparable to the amount of non-chimeric transgenic plants of the Triticum genus, which are regenerated in a corresponding comparative method, which is different in that selection of a transformed cell takes place based on a property mediated by the genetic component or a portion thereof, i.e., based on at least one selection marker. This is also surprising because those skilled in the art would expect a much lower amount of non-chimeric transgenic plants of the Triticum genus based on experience with methods for marker gene-free production of transgenic maize plants, for example. Because of the great amount of effort involved in the creation of selection marker-free plants of the Triticum genus with the subsequent removal of the selection marker gene, as described above, those skilled in the art will still regard the method according to the invention as being advantageous and superior to the state of the art if the amount of non-chimeric transgenic plants of the Triticum genus from step (b) is lower than that in the comparative method. The amount may be lower by a factor of max. 10, by a factor of max. 9, by a factor of max. 8, by a factor of max. 7, by a factor of max. 6, by a factor of max. 5, by a factor of max. 4.5, by a factor of max. 4, by a factor of max. 3.5, by a factor of max. 3, by a factor of max. 2.5 or by a factor of max. 2.

As described above, chimeric transgenic plants may occur when regenerating shoot has been formed from multiple original cells, wherein some of these cells were transgenic, but others were not transgenic. For example, sectoral chimers or periclinal chimers may be formed. Due to the amount of non-transgenic tissue in the chimeric plants, these can be identified, for example, by quantitative PCR (Faize et al., 2010).

Another method of detection for chimeric transgenic plants is analysis of the first progeny of a starting transformant. The genetic component or a portion thereof introduced into the starting transformant can be transmitted to the next generation according to Mendel's laws. In integration of a copy of the genetic component or a portion thereof into the genome of the plant cell, this is integrated into only one chromosome of the diploid genome. In a non-chimeric plant, the genetic component or a portion thereof will then be found in 50% of the resulting gametes in meiosis. However, in chimeric transgenic plants, gametes are also formed form the non-transgenic portions of the plant. Only gametes that do not contain the genetic component or a portion thereof are formed in these tissues. The amount of non-transgenic gametes is thus increased to >50% in chimeric transgenic plants, as seen for the whole plant. In the selfing progeny of the chimeric starting transformants, the amount of non-transgenic progeny is thus increased >25%, which is thus greater than would be expected according to Mendel's laws. One example of the segregation that does not follow Mendel's laws in the first filial generation of a chimeric transgenic plant is given by Coussens et al. (2012).

In another particularly preferred embodiment of the method according to the invention, the amount of chimeric transgenic plants of the Triticum genus from step (b) is comparable to the amount of chimeric transgenic plants of the Triticum genus regenerated in a corresponding comparative method, which is different in that there is a selection of a transformed cell based on a property mediated by the genetic component or a portion thereof, i.e., based on at least one selection marker. This is also surprising because, based on experience with methods of marker gene-free production of transgenic maize plants, for example, those skilled in the art would expect a much higher proportion of chimeric transgenic plants of the Triticum genus. Because of the great amount of labor involved in the creation of selection marker-free plants of the Triticum genus with the subsequent removal of the selection marker gene as described above, those skilled in the art would regard the method according to the invention as advantageous and also superior to the state of the art even if the amount of chimeric transgenic plants of the Triticum genus from step (b) is greater than that in the comparative method. The amount may be greater by a factor of max. 10, by a factor of max. 8, by a factor of max. 6, by a factor of max. 5, by a factor of max. 4, by a factor of max. 3.5, by a factor of max. 3, by a factor of max. 2.5, by a factor of max. 2, by a factor of max. 1.8, by a factor of max. 1.6, by a factor of max. 1.4, by a factor of 1.2 or by a factor of max. 1.1.

In a particularly preferred embodiment, the method according to the invention is characterized in that it includes after step (b) another step (c) selection of the regenerated transgenic plant from step (b). The selection is preferably based on the molecular structure of the genetic component or a portion thereof or based on the property, in particular a phenotypic property, which is mediated by the genetic component directly or indirectly (e.g., herbicide resistance, pathogen resistance, height of growth, yield, leaf structure). Molecular structure of the genetic component or a portion thereof refers in particular to the sequential sequence of nucleotides of the genetic component or a portion thereof. Step (c) serves to detect successful transformation of the genetic component or a portion thereof into the cell of a plant of the Triticum genus, i.e., including the transfer of the genetic component or a portion thereof into the genome of the plants. Those skilled in the art therefore have access to numerous different methods of molecular biology known from the state of the art. Thus detection of the genetic component introduced into the cell is possible, for example, by means of a polymerase chain reaction (Mullis, 1988), by hybridization of a detectable single strand nucleic acid which is complementary to the genetic component having been introduced, with the genomic DNA of the transgenic plants, e.g., in the so-called Southern Blot (Southern, 1975) or by sequencing the genome of the transgenic plant (Kovalic et al., 2012). In addition the molecular structure of the genetic component or a portion thereof may also refer to the molecular structure of a derived component which is obtained, for example, by transcription, processing and/or translation from the genetic component. Thus, detection of the transcript or the coded peptide/polypeptide/protein of the genetic component thereby introduced or a portion thereof in the transgenic plant is also considered to be proof of successful transformation of the genetic component or a portion thereof, i.e., suitable for selection. Examples of methods with which those skilled in the art are familiar and which can be used for the purpose of detection of the transcript include: transcription of RNA formed from the genetic component or a portion thereof to cDNA and subsequent polymerase chain reaction (RT-PCR; Sambrook et al., 2001), hybridization of a detectable single strand nucleic acid which is complementary to the genetic component introduced, with the RNA of the transgenic plant (Northern blot, Sambrook et al., 2001) or transcription of RNA formed from the genetic component or a portion thereof to cDNA and subsequent sequencing of the entire pool of cDNA thereby obtained. The coded peptide/polypeptide/protein can be identified, for example, by means of immunodetection or by various methods such as Western Blot or ELISA. Furthermore, a phenotypic property, which is mediated directly or indirectly by the genetic component can be detected for selection. Such a phenotypic detection may also include detection of a modified chemical composition of the plant cell. This modified chemical composition can then be detected by means of known methods of chemical analysis.

In another particularly preferred embodiment of the method according to the invention, the at least one cell of a plant of the Triticum genus is transformed with the complete genetic component in step (a), in particular undergoing a stable transformation. “Complete” preferably means that at least one cell of a plant of the Triticum genus is transformed with the genetic component, wherein the genetic component has not undergone any truncation (for example, from the 5′- or 3′-end) that would impair the intended functionality of the genetic component in the cell of a plant of the Triticum genus and in particular preferably that the at least one cell of a plant of the Triticum genus has been transformed with all the nucleotides of the genetic component.

In another particularly preferred embodiment of the method according to the invention, after the transformation in step (a), the genetic component has an expression level in the cell of a plant of the Triticum genus after transformation that permits the intended functionality of the genetic component. The method according to the invention is preferably characterized in that 10%, 20%, 30%, 40%, 50%, 60%, 70%, 75%, 80%, 85%, 90%, 95% or 100% of the transformed cells from step (a) have a detectable expression level, preferably an expression level which enables the intended functionality of the genetic component, or that 10%, 20%, 30%, 40%, 50%, 60%, 70%, 75%, 80%, 85%, 90%, 95% or 100% of the regenerated transgenic plant of the Triticum genus from step (b) includes cells having a detectable expression level, preferably an expression level which enables the intended functionality of the genetic component.

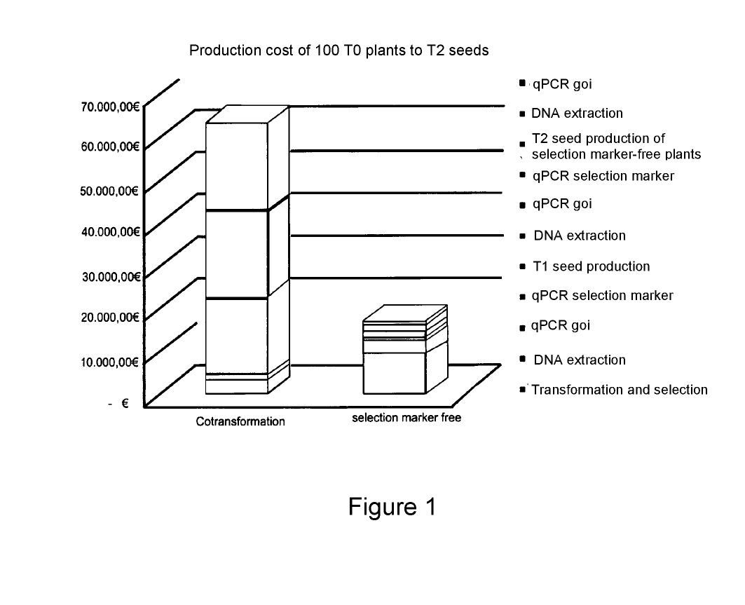

Methods described above for producing a transgenic plant of the Triticum genus can be used advantageously because selection marker-free transgenic lines of a high quality can be developed from the transgenic plants. To obtain transgenic lines of a comparable quality, the only possibility available at the present time would be to create selection marker-free plants by means of co-transformation and subsequent segregation. If the effort required to generate a selection marker-free transgenic line via the co-transformation batch is compared with the effort required by a method according to the present invention, then the cost of development of a homozygotic selection marker-free transgenic line would be approximately 50 times greater. FIG. 1 shows an estimate of the cost of generating 100 T₀ transgenic lines in a co-transformation batch and with the selection marker-free transformation. The starting transformants generated will be analyzed further in the next generations with the goal of obtaining homozygotic selection marker-free seed pools. Whereas the yield of single-copy selection marker-free lines in the co-transformation batch starting from 100 initial transformants in the selection marker-free transformation according to the invention, 30 homozygotic seed pools can be expected, whereas the yield of single-copy selection marker-free lines in the co-transformation batch would be only two homozygotic seed pools due to the fact that the co-transformation rate is only 30% to 50% and due to the requirement that both the gene of interest and the selection marker must be present as a single-copy event in order to obtain a sufficiently high probability of segregation of the two transgenes.

The present invention also relates to a transgenic plant of the Triticum genus which was produced by one of the methods described above and a progeny, a portion or a seed thereof wherein the progeny, the portion or the seed thereof contains the genetic component that was transmitted as a transgene in step (a) of the method according to the invention. A portion here may refer to a cell, a tissue or an organ.

Some of the terms used in this patent application will be described first in greater detail below:

A “gene of interest” may refer to any type of DNA or RNA molecule which codes for a protein, for example, or a nucleic acid molecule.

A “plant of the Triticum genus” refers, for example, to a plant of the species Triticum aestivum, a plant of the species Triticum durum or a plant of the species Triticum spelta.

A “regulatory sequence” in conjunction with the present invention refers to a nucleic acid sequence which controls the expression of a gene of interest. Examples include promoters, operators, enhancer elements, attenuators, cis elements, etc.

The term “selection marker” in conjunction with the present invention is understood to be equivalent to “selection marker gene” or “marker gene.” Examples of selection markers that can be used have already been described above.

“Transformation efficiency” may refer to the ratio of the number of explants having positive transgenic shoots to the number of initial explants. The transformation efficiency is preferably given as a percentage.

The term “comparable” in conjunction with two or more numerical amounts being compared means that the amounts differ from one another by at most ±5%.

Embodiments and forms of the present invention are described below in an exemplary manner with reference to the accompanying figures and sequences:

FIG. 1. Cost comparison for the generation of 100 T₀ plants by co-transformation (left) and by a method according to the present invention and additional identification of homozygotes, selection marker-free seed pools.

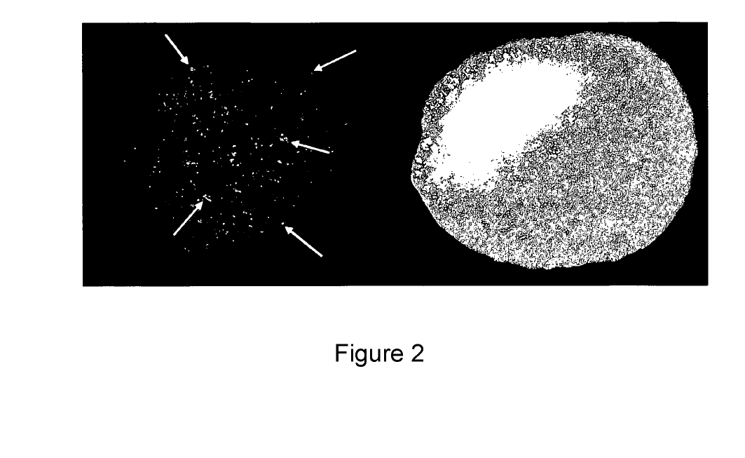

FIG. 2. View of the scutellum of a tDT-transformed wheat embryo 5 days after infection with A. tumefaciens (left: in fluorescent light; right: in daylight); arrows show fluorescent regions in the initial explant, which give an exemplary indication of the agrobacteria.

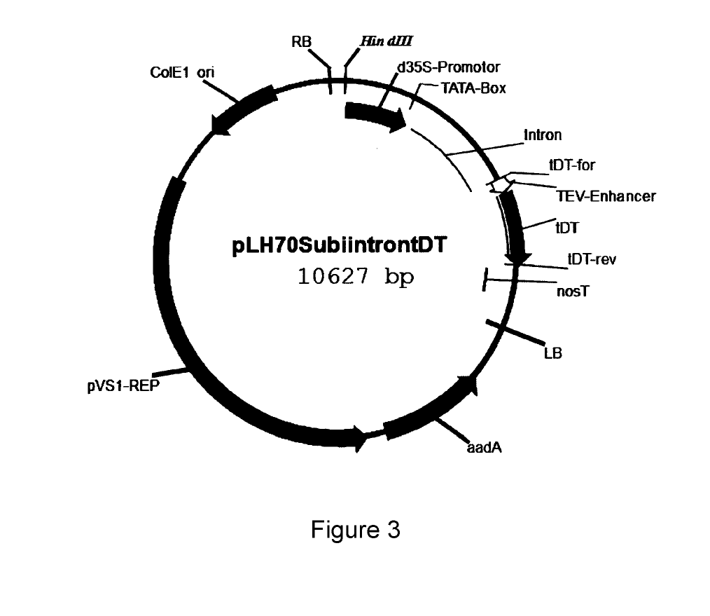

FIG. 3. Binary vector pLH70SubiintrontDT (tDT is tandem dimer tomato, a red fluorescent protein).

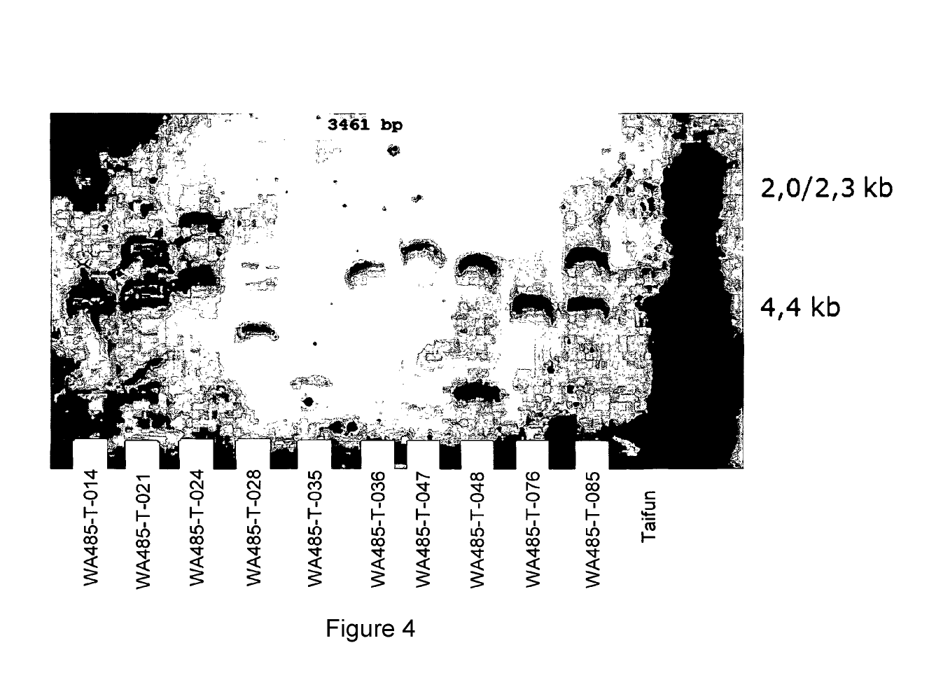

FIG. 4. Southern Blot of selection marker-free transgenic lines of the transformation experiment WA1; 20 μg of the genomic DNA of the respective line was digested completely with the Hindlll enzyme, separated in 0.8% agarose gel, blotted on a nylon membrane and then hybridized with a DIG-labeled PCR product (tDT-ref/tDT-for).

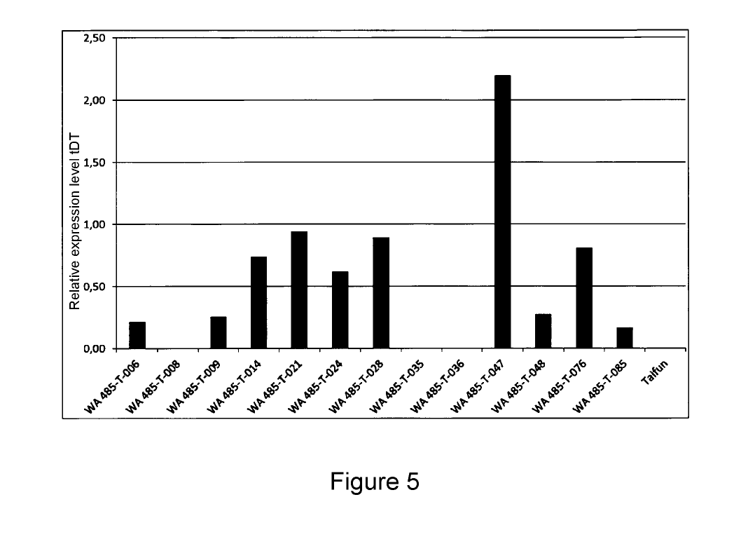

FIG. 5. Expression analysis of the tDT gene introduced using qRT-PCR in selected transgenic wheat plants.

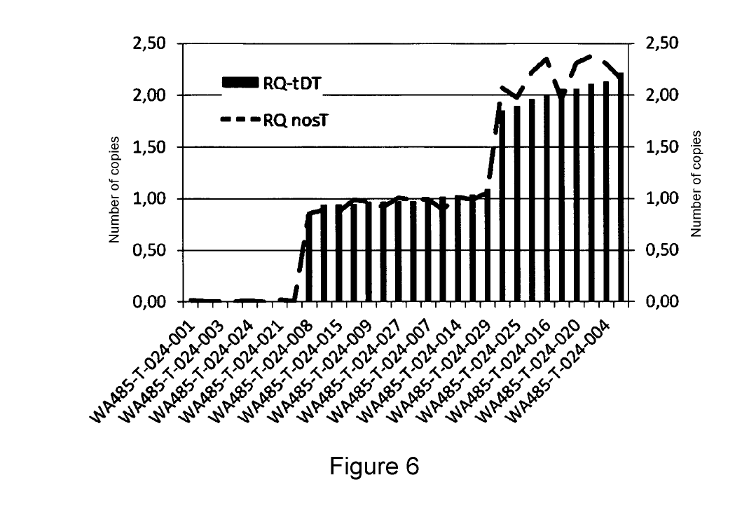

FIG. 6. Determination of the zygotism status by means of qPCR on the transgene tDT introduced as well as the nos terminator introduced (see FIG. 3).

Selection marker-free transformation of wheat plants of the Taifun variety:

Wheat plants of the Taifun variety were cultured in a greenhouse. The cultivation conditions were: 18° C. by day and 16° C. at night with a day length of 16 hours. The light sources were sodium lamps (Maaster SON-T Agro 400W). The size of the embryos in the developing ears of wheat was tested regularly, such that ears containing the grains with embryos approximately 1.5-2.5 mm in size were harvested and stored standing in water at 4° C. in the dark until further use.

In preparation for the isolation of the immature wheat embryos, the grains were isolated from the wheat ears and then sterilized superficially. To do so, the grains were first incubated for 45 seconds in 70% ethanol and then incubated for 10 minutes in 1% sodium hypochloride solution. After sterilization, the grains were freed of any adhering sodium hypochloride by washing with sterile water several times. The sterilized grains were then stored at 4° C. in the dark until further use.

Agrobacterium tumefaciens was cultured for transformation by starting with a glycerin culture of A. tumefaciens strain AGL1, which carries the gene construct to be transformed in the pLH70SubiintrontDT binary vector (FIG. 3). After spreading on a selective LB medium (with 100 mg/L rifampicin, 100 mg/L carbenicillin, 50 mg/L spectinomycin, 25 mg/L streptinomycin), a 2 mL liquid culture in mg/L medium (Wu et al., 2009) containing 100 mg/L rifampicin, 100 mg/L carbenicillin, 50 mg/L spectinomycin, 25 mg/L streptinomycin was inoculated with a single colony and cultured overnight at 28° C. and 200 rpm. The next day, 250 μL of the liquid culture was used for inoculating 50 mL fresh mg/L medium (100 mg/L rifampicin, 100 mg/L carbenicillin, 50 mg/L spectinomycin, 25 mg/L streptinomycin) and the culture was cultured overnight at 28° C. and 200 rpm. One aliquot of the overnight culture was then centrifuged (5 minutes at 4° C. and 3500 g′s), the supernatant was discarded and the bacteria pellet was resuspended in an equal volume of Inf liquid medium (Table 1) with 100 μM acetosyringone. The agrobacterium suspension prepared in this way was the used for infection of the immature embryos.

The immature embryos were isolated from the sterilized wheat grains and collected in the Inf liquid medium (Table 1). The embryos were THEN washed once with fresh Inf liquid medium and then pretreated by centrifuging at 15,000 rpm for 10 minutes. For infection with the agrobacteria, the prepared agrobacteria suspension was applied to the embryos and the embryos were then shaken for 30 seconds in the agrobacteria suspension. Following that the embryos were incubated for 5 minutes more at room temperature in the agrobacteria suspension. The immature embryos were then applied to co-cul medium (Table 1) with the scutellum side facing up. The explants treated in this way were incubated for 2 days at 23° C. in the dark. FIG. 2 shows the scutellum of a transformed wheat embryo several days after infection with A. tumefaciens. Wheat embryos were transformed with a reporter gene construct, which triggers the formation of a red fluorescent protein in the transformed cells. The figure at the left shows the scutellum in daylight while the figure at the right shows the scutellum under fluorescent light. It can be seen clearly that most of the cells of the scutellum are expressing the transgene and have thus been successfully infected with A. tumefaciens.

After 2 days of co-culture of the immature wheat embryos with agrobacteria, the embryonic axis was removed from each embryo using a sharp scalpel, and the remaining scutella were placed on a resting medium (Table 1). The plates with the scutella were then incubated for 5 days at 25° C. in the dark. Next the resulting callus was subcultured for 21 days on the resting medium at 25° C. in the dark (Table 1).

The induced callus was transferred entirely to LSZ medium (Table 1) and placed in the light for 14 days. The resulting green shoots were separated from the callus and transferred to LSF medium (Table 1) for rooting. The shoots were separated from one another as much as possible to obtain single shoots. Shoots originating from an original explant (scutellum) were kept together in this process. After sufficient growth in length of the shoots, leaf samples could be taken from them for extraction of DNA and then for subsequent PCR analyses.

TABLE 1
Composition of the media used

| Inf liquid medium | | | Co-Cul medium | | | resting medium | | |
|---|---|---|---|---|---|---|---|---|
| 1/10 x | | MS inorganic salts | 1/10 x | | MS inorganic | 1x | | MS inorganic |
| | | | | | salts | | | salts |
| 1X | | MS vitamins | 1X | | MS vitamins | 1X | | MS vitamins |
| 10 | g/L | glucose | 10 | g/L | glucose | 40 | g/L | maltose |
| 0.5 | g/L | MES | 0.5 | g/L | MES | 0.5 | g/L | glutamine |
| | | | 100 | μM | acetosyringone | 0.1 | g/L | casein |
| | | | | | | | | hydrolysate |
| | | | 5 | μM | silver nitrate | 0.75 | g/L | MgCL2 × 7H2O |
| | | | 5 | μM | copper sulfate | 1.95 | g/L | MES |
| | | | 8 | g/L | agarose | 100 | mg/L | ascorbic acid |
| | | | | | | 150 | mg/L | Timentin |
| | | | | | | 2.2 | mg/L | Pictoram |
| | | | | | | 0.5 | mg/L | 2,4-D |
| | | | | | | 2 | g/L | Gelrite |

| | |
|---|---|
| LSZ medium | LSF medium |

| 1x | | LS inorganic salts | 1x | | LS inorganic salts |
|---|---|---|---|---|---|
| 1X | | LS vitamins | 1X | | LS vitamins |
| | | (Ishida et al., | | | (Ishida et al., |
| | | 2007) | | | 2007) |
| 20 | g/L | sucrose | 15 | g/L | sucrose |
| 0.1 | mM | Fe-EDTA | 0.1 | mM | Fe-EDTA |
| 5 | mg/L | zeatin | 0.2 | mg/L | indole butyric acid |
| 10 | μM | copper sulfate | 10 | μM | copper sulfate |
| 0.5 | g/L | MES | 0.5 | g/L | MES |
| 150 | mg/L | Timentin | 150 | mg/L | Timentin |
| 8 | g/L | agar | 3 | g/L | Gelrite |

Results:

Three independent transformation experiments were performed on Triticum aestivum as described above without using a selection marker. In all three experiments, transgenic plants were obtained without using selection markers (see Table 2). The high number of explants yielding transgenic shoots was surprising. In the WA1 experiment, of the 151 embryos infected, 89 were stimulated to regeneration of shoots. The regenerated shoots were first combined to for a total of 341 shoot pools for the PCR analysis. To do so, two to three shoots of an explant, the number depending on the number of regenerated shoots per starting explant, were combined in a sample vessel for the purpose of DNA extraction. If more than three shoots were to be regenerated per starting explant, then several shoots would be prepared from one starting explant. However, leaf samples of shoots of multiple starting explants were never combined. Of the shoot pools that were analyzed, a surprisingly high number were positive (78 or ˜23%). Of the 341 shoot pools of the 89 explants, 78 transgenic shoot pools from 48 explants were identified. The 111 shoots forming the basis of the 78 shoot pools were then sampled individually and tested again for the presence of the transgene.

To detect the transgene in the regenerated shoots, the DNA isolated from the shoot pools or from the individual shoots was tested by PCR for the presence of recombinant DNA. The tDT-1 primer (SEQ ID NO. 1) and tDT-2 (SEQ ID NO. 2) were used to do so. DNAs in which a 287 by fragment was amplified showed the presence of the recombinant DNA that had been introduced and were considered to be transgenes. To determine the number of copies of the transgene introduced into the wheat germ, a quantitative PCR was performed using the primers nosTxxxf01 (SEQ ID NO. 3) and nosTxxxr03 (SEQ ID NO. 4) as well as the probe nosTXXXMGB (SEQ ID NO. 5). Quantitative PCR confirmed the results obtained previously with traditional PCR.

In the WA1 experiment, the transgene was detected in a total of 82 shoots. The 82 shoots originated from 37 explants/embryos that were initially infected with A. tumefaciens. Thus, despite the omission of the marker gene-based selection, a transformation efficiency of approximately 25% was achieved in the WA1 experiment. This efficiency is calculated from the 37 explants having positive shoots of the 151 explants used originally.

In the WA2 and WA3 experiments, all the single shoots regenerated from the explants were tested by PCR because a surprisingly high yield of transgenic shoots was obtained in the WA1 experiment and thus the use of the pool PCR strategy was superfluous. In a direct analysis of the regenerated shoots, transgenic single shoots were identified in 56% (WA2) and 75% (WA3) of the regenerable explants.

If the efficiency of transformation is calculated, based on the number of the starting explants used, this yields a transformation efficiency of 27% for experiment WA2 and 40% for experiment WA3.

Averaging over all three transformation experiments in the case of Triticum aestivum without using marker gene-based selection reveals that an average of 55% of the regenerable explants produced transgenic single shoots and an average transformation efficiency of approximately 30% was achieved.

In parallel, the control experiments WA1 K, WA2K and WA3K were carried out in which the hygromycin phosphotransferase (hpt) selection marker was integrated into the genome of the Taifun wheat variety together with the gene of interest. The transformations were performed as described in EP 2 460 402, i.e., hygromycin was added to the medium during the callus and regeneration phases in concentrations of 15 mg/L and 30 mg/L, respectively.

In the WAK1 experiment, a transformation efficiency of 37% was achieved (75 explants with positive shoots of 204 starting explants). In the WAK2 experiment the transformation efficiency was 24% (37 explants with positive shoots of 153 starting explants) and in the WAK3 experiment the transformation efficiency was 27% (47 explants with positive shoots of 175 starting explants). Thus, on the average, an efficiency of 30% (ø WAK) was achieved in these transformation experiments.

The transformation efficiency found here that, without using selection, this corresponds to the efficiency usually achieved in wheat transformation experiments with marker gene-based selection, and in some cases the efficiency seemed to be even higher.

TABLE 2
Results of three transformation experiments without using a marker gene-based selection
in Triticum aestivum (Taifun variety); WAKx denotes the control experiment with marker
gene-based selection, WAx denotes experiments without marker-based gene selection

| | | | PCR analysis | | | |
|---|---|---|---|---|---|---|
| | | (B) | (C) | (D) | (E) | (F) |
| | (A) | Explants | Number of | Number of | Number of | Transformation |
| Experiment- | Starting | with | shoots | positive | explants with | efficiency = |
| No. | explant | regeneration | analyzed | shoots | positive shoots | (E)/(A) in % |
| WAK1 | 204 | — | — | — | 75 | 37% |
| WAK2 | 153 | — | — | — | 37 | 24% |
| WAK3 | 175 | — | — | — | 47 | 27% |
| Ø WAK | 532 | — | — | — | 159 | 30% |
| WA1 | 151 | 89 | 341 | 82 | 37 | 25% |
| WA2 | 100 | 48 | 396 | 57 | 27 | 27% |
| WA3 | 106 | 56 | 406 | 73 | 42 | 40% |
| Ø WA | 357 | 193 | 1143 | 212 | 106 | 30% |

Detection of the transgenic nature of the selection marker-free transgenic lines created in this way was obtained by qPCR, as described above. At the same time this analysis permits an estimate of the amount of single-copy lines, which are of particular interest for further use for commercial purposes. Here again, it is found that the results do not show a difference between transformations with and without use of a selection marker.

Thus, 12 independent single-copy lines were identified in the WA2 experiment, based on the qPCR batch. Since a total of 27 independent transgenic events were generated, this corresponds to a rate of 44% single-copy events. In the WA3 experiment, 12 independent single-copy events were also generated, which corresponds to a rate of 29% with a total of 42 independent events generated.

To further verify the transgenic property of the lines thus created, a Southern Blot test was performed on selected T₀ plants of the WA1 experiment. Those skilled in the art are aware of the fact that when the T-DNA is transferred from the agrobacterium to the plant genome, in many cases only shortened T-DNA fragments are transferred. These are deleted on the LB (left border) side. Therefore, T-DNAs for use in a transformation with marker gene are frequently designed so that the selection marker used for the selection is positioned on the LB side of the T-DNA. Then only events with complete T-DNA, i.e., when the marker gene is completely transferred, are selected. Since only the gene of interest is present as T-DNA in marker gene-free transformation, the gene of interest could thus be involuntarily shortened in the transfer, which usually results in defective expression of the transferred gene of interest in the plant genome.

To test the transferred T-DNA for thoroughness, hybridization experiments were conducted. In these experiments the introduced tDT gene was used as the hybridization probe. The genomic DNA was digested with Hindlll, so that a completely integrated T-DNA would yield a hybridization fragment of more than 3.0 kb. As shown in FIG. 4, a hybridization fragment was found in all of the PCR-positive lines tested. Genomic DNA of the negative control (Taifun) would not hybridize with the probe. Since all the resulting hybridization fragments are >3.0 kb in size, it has thus been demonstrated that the T-DNA in all lines created is completely integrated. This shows that the quality of the transgene after transfer is comparable to that when using a transformation with marker gene from the LB side. For those skilled in the art, this was to be expected.

In addition, the transgenic lines produced using the marker gene-free transformation method were tested in greater detail with regard to the level of expression of the integrated transgene. When using T-DNA with selection marker, for successful selection of transgenic lines, it is necessary for the gene of the selection marker to be expressed and thus for the functional protein to be formed. T-DNA integration in genomic regions that do not permit any reading of the gene construct introduced therefore cannot be identified as a transgenic line. When using the selection marker-free transformation, events integrated into regions of the genome that do not allow reading of the transgene are also identified as a transgenic line by means of the methods of molecular biology such as PCR. There is thus the risk that an increased amount of transgenic lines having no expression of the transgene that has been introduced may be produced.

Therefore, the level of expression of the transgene that was introduced is determined from randomly selected lines of the transformation experiment WA1 by means of qRT-PCR (FIG. 5). No expression of the transgene was detected in only 3 of the 13 transgenic lines that were analyzed. All other lines showed a definite expression of the transgene, although the level of expression was definitely different among the individual lines. However, this is also the case in transgenic lines transformed by using a selection marker. Thus there are also no differences in the quality of the transgene between transgenic lines created with the help of a selection marker and those without a selection marker.

To detect the formation of chimeric transgenic plants, the question of whether the transgene introduced is transmitted to the next generation according to Mendel's laws was investigated. To do so, the seeds from six transgenic lines were laid out (30 grains per line) and the presence of the transgene and its zygote status were determined by qPCR on the transgene tDT introduced as well as on the nos-terminator that was introduced. FIG. 6 shows the result of an analysis of a progeny as an example. A 1:2:1 heredity pattern for a monogenic heredity model is clearly observable. Table 3 shows a summary of the results of the progeny analysis.

Five of the six progeny analyzed show a heredity according to Mendel's laws (corresponding to 83%). It can thus be assumed that most of the transgenic starting transformants created were homogeneous with respect to the transgene. On the one hand, the non-Mendelian succession with the transgenic line WA1-T-014 can be attributed to a non-homogenous, i.e., chimeric transgenic plant, but on the other hand, the integration of the transgene into an important gene of the plant may also occur. Therefore there are partially lethal plants/embryos, which would also explain the poor germination capacity of this progeny (only 20 of 30 grains would germinate).

TABLE 3
Results of a progeny analysis for detection
of chimeric transgenic wheat plants

| Transgenic | Azy- | Hemi- | Homo- | | Split | |
|---|---|---|---|---|---|---|
| line | gotic | zygotic | zygotic | Total | ratio | X² |
| WA1-T-006 | 8 | 16 | 3 | 27 | 1:2:1 | 0.25 |
| WA1-T-008 | 8 | 14 | 7 | 29 | 1:2:1 | 0.95 |
| WA1-T-009 | 4 | 16 | 9 | 29 | 1:2:1 | 0.36 |
| WA1-T-014 | 11 | 6 | 3 | 20 | ? | 0.01 |
| WA1-T-024 | 8 | 13 | 9 | 30 | 1:2:1 | 0.74 |
| WA1-T-028 | 9 | 17 | 4 | 30 | 1:2:1 | 0.33 |

### REFERENCES

Ahmad, R., Kim, Y. H., Kim, M. D., Phung, M. N., Chung, W. I., Lee, H. S., . . . & Kwon, S. Y. (2008). Development of selection marker-free transgenic potato plants with enhanced tolerance to oxidative stress. Journal of Plant Biology, 51(6), 401-407.

Amoah, B. K., Wu, H., Sparks, C., & Jones, H. D. (2001). Factors influencing Agrobacterium-mediated transient expression of uidA in wheat inflorescence tissue. Journal of Experimental Botany, 52(358), 1135-1142.

Ballester, A., Cervera, M., & Peña, L. (2010). Selectable marker-free transgenic orange plants recovered under non-selective conditions and through PCR analysis of all regenerants. Plant Cell, Tissue and Organ Culture (PCTOC), 102(3), 329-336.

Broothaerts, W., Mitchell, H. J., Weir, B., Kaines, S., Smith, L. M., Yang, W., . . . & Jefferson, R. A. (2005). Gene transfer to plants by diverse species of bacteria. Nature, 433(7026), 629-633.

Chang, H. H., & Chan, M. T. (1991). Improvement of potato (Solanum tuberosum L.) transformation efficiency by Agrobacterium in the presence of silver thiosulfate. Bot. Bull. Acad. Sin, 32, 63-70.

Cheng, M., Fry, J. E., Pang, S., Zhou, H., Hironaka, C. M., Duncan, D. R., . . . & Wan, Y. (1997). Genetic transformation of wheat mediated by Agrobacterium tumefaciens. Plant Physiology, 115(3), 971-980.

Comai, L., Sen, L. C., & Stalker, D. M. (1983). An altered aroA gene product confers resistance to the herbicide glyphosate. Science, 221(4608), 370-371.

Coussens, G., Aesaert, S., Verelst, W., Demeulenaere, M., De Buck, S., Njuguna, E., . . . & Van Lijsebettens, M. (2012). Brachypodium distachyon promoters as efficient building blocks for transgenic research in maize. Journal of experimental botany, 63(11), 4263-4273.

De Block, M., Botterman, J., Vandewiele, M., Dockx, J., Thoen, C., Gossele, V., . . . & Leemans, J. (1987). Engineering herbicide resistance in plants by expression of a detoxifying enzyme. The EMBO journal, 6(9), 2513.

De Vetten, N., Wolters, A. M., Raemakers, K., van der Meer, I., ter Stege, R., Heeres, E., . . . & Visser, R. (2003). A transformation method for obtaining marker-free plants of a cross-pollinating and vegetatively propagated crop. Nature biotechnology, 21(4), 439-442.

Erikson, O., Hertzberg, M., & Näsholm, T. (2005). The dsdA gene from Escherichia coli provides a novel selectable marker for plant transformation. Plant molecular biology, 57(3), 425-433.

Faize, M., Faize, L., & Burgos, L. (2010). Using quantitative real-time PCR to detect chimeras in transgenic tobacco and apricot and to monitor their dissociation. BMC biotechnology, 10(1), 53.

Ferradini, N., Nicolia, A., Capomaccio, S., Veronesi, F., & Rosellini, D. (2011). Assessment of simple marker-free genetic transformation techniques in alfalfa. Plant cell reports, 30(11), 1991-2000.

Frame, B. R., Shou, H., Chikwamba, R. K., Zhang, Z., Xiang, C., Fonger, T. M., . . . & Wang, K. (2002). Agrobacterium tumefaciens-mediated transformation of maize embryos using a standard binary vector system. Plant Physiology, 129(1), 13-22.

Hiei, Y., Ohta, S., Komari, T., & Kumashiro, T. (1994). Efficient transformation of rice (Oryza sativa L.) mediated by Agrobacterium and sequence analysis of the boundaries of the T-DNA. The Plant Journal, 6(2), 271-282.

Hiei, Y., Ishida, Y., Kasaoka, K., & Komari, T. (2006). Improved frequency of transformation in rice and maize by treatment of immature embryos with centrifugation and heat prior to infection with Agrobacterium tumefaciens. Plant cell, tissue and organ culture, 87(3), 233-243.

Hensel, G., Kastner, C., Oleszczuk, S., Riechen, J., & Kumlehn, J. (2009). Agrobacterium-mediated gene transfer to cereal crop plants: current protocols for barley, wheat, triticale, and maize. International Journal of Plant Genomics, 2009.

Ishida, Y., Saito, H., Hiei, Y., & Komari, T. (2003). Improved protocol for transformation of maize (Zea mays L.) mediated by Agrobacterium tumefaciens. Plant Biotechnology, 20(1), 57-66.

Ishida, Y., Hiei, Y., & Komari, T. (2007). Agrobacterium-mediated transformation of maize. Nature protocols, 2(7), 1614-1621.

Joersbo, M. (2001). Advances in the selection of transgenic plants using non-antibiotic marker genes. Physiologia plantarum, 111(3), 269-272.

Kortstee, A. J., Khan, S. A., Helderman, C., Trindade, L. M., Wu, Y., Visser, R. G. F., . . . & Jacobsen, E. (2011). Anthocyanin production as a potential visual selection marker during plant transformation. Transgenic research, 20(6), 1253-1264.

Kovalic, D., Garnaat, C., Guo, L., Yan, Y., Groat, J., Silvanovich, A., . . . & Bannon, G. (2012). The Use of Next Generation Sequencing and Junction Sequence Analysis Bioinformatics to Achieve Molecular Characterization of Crops Improved Through Modern Biotechnology. The Plant Genome, 5(3), 149-163.

Kraus, J. (2010). Concepts of marker genes for plants. In Genetic Modification of Plants (pp. 39-60). Springer Berlin Heidelberg.

Li, B., Xie, C., & Qiu, H. (2009). Production of selectable marker-free transgenic tobacco plants using a non-selection approach: chimerism or escape, transgene inheritance, and efficiency. Plant cell reports, 28(3), 373-386.

Liu, X., Jin, W., Liu, J., Zhao, H., & Guo, A. (2011). Transformation of wheat with the HMW-GS 1Bx14 gene without markers. Russian Journal of Genetics, 47(2), 182-188.

Maluszynski, M. (Ed.). (2003). Doubled haploid production in crop plants: a manual. Springer.

Mullis, K. B., & Erlich, H. A. (1988). Primer-directed enzymatic amplification of DNA with a thermostable DNA polymerase. Science, 239(4839), 487-491.

Muβmann, V., Serek, M., & Winkelmann, T. (2011). Selection of transgenic Petunia plants using the green fluorescent protein (GFP). Plant Cell, Tissue and Organ Culture (PCTOC), 107(3), 483-492.

Potrykus, I., Bilang, R., Futterer, J., Sautter, C., Schrott, M., & Spangenberg, G. (1998). Genetic engineering of crop plants. Agricultural Biotechnology, 119-159.

Reed, J., Privalle, L., Powell, M. L., Meghji, M., Dawson, J., Dunder, E., . . . & Wright, M. (2001). Phosphomannose isomerase: an efficient selectable marker for plant transformation. In Vitro Cellular & Developmental Biology-Plant, 37(2), 127-132.

Sambrook, J., Russell, D. W., & Russell, D. W. (2001). Molecular cloning: a laboratory manual (3-volume set).

Shrawat, A. K., & Lörz, H. (2006). Agrobacterium-mediated transformation of cereals: a promising approach crossing barriers. Plant Biotechnology Journal, 4(6), 575-603.

Southern, E. M. (1975). Detection of specific sequences among DNA fragments separated by gel electrophoresis. Journal of molecular biology, 98(3), 503-517.

Tuteja, N., Verma, S., Sahoo, R. K., Raveendar, S., & Reddy, I. B. L. (2012). Recent advances in development of marker free transgenic plants: regulation and biosafety concern. Journal of Bioscience, 37(1), 162-197.

Vasil V, V Srivastava, A. M. Castillo, M. E. Fromm, I. K. Vasil (1993) Rapid production of transgenic wheat plants by direct bombardment of cultured immature embryos. Bio/Technology 11:1553-1558.

Wang, Y. L., Xu, M. X., Yin, G. X., Tao, L. L., Wang, D. W., & Ye, X. G. (2009). Transgenic wheat plants derived from Agrobacterium-mediated transformation of mature embryo tissues. Cereal Research Communications, 37(1), 1-12.

Wu, H., Doherty, A., & Jones, H. D. (2009). Agrobacterium-mediated transformation of bread and durum wheat using freshly isolated immature embryos. In Transgenic wheat, barley and oats (pp. 93-103). Humana Press.

Zhao, Z. Y., Gu, W., Cai, T., Tagliani, L., Hondred, D., Bond, D., . . . & Pierce, D. (2002). High throughput genetic transformation mediated by Agrobacterium tumefaciens in maize. Molecular Breeding, 8(4), 323-333.

WO 1998/054961 A2 (Novartis AG et al.) “Plant transformation methods”

WO 2002/012520 A1 (Japan Tobacco Inc.) “Method of improving gene transfer efficiency into plants cells”

WO 2005/017152 A1 (Japan Tobacco Inc. et al.) “Method for improving plant transformation efficiency by adding copper ion”

WO 2005/017169 A1 (Japan Tobacco Inc. et al.) “Method of transducing gene into plant material”

WO 2007/069643 A1 (Japan Tobacco Inc. et al.) “Method for Improving Transformation Efficiency Using Powder”

WO 2008/028121 A1 (Monsanto Technology LLC et al.) “Methods for producing transgenic plants”

EP 0 672 752 B1 (Japan Tobacco Inc.) “Method of transforming monocotyledons using scutella of immature embryos”

EP 2 274 973 A1 (Japan Tobacco Inc.) “Agrobacterium-mediated method for producing transformed plant”

EP 2 460 402 A1 (Japan Tobacco Inc.) “Method for gene transfer into Triticum plants using Agrobacterium bacterium, and Method for production of transgenic plant of triticum plant”

US 2011/0030101 A1 (Japan Tobacco Inc. et al.) “Agrobacterium-mediated method for producing transformed plant”

1. A method of producing a transgenic plant of the Triticum genus, comprising the steps of:
(a) transforming at least one cell of a plant of the Triticum genus with a genetic component by co-culturing cells of an explant of said plant with at least one bacterial cell from the Rhizobiaceae family, wherein said bacterial cell comprises the genetic component, and
(b) regenerating a transgenic plant of the Triticum genus from at least one transformed cell or from at least one transformed cell derived from at least one transformed cell produced in step (a),
wherein in step (a) and step (b), there is no selection of the at least one transformed cell based on a property imparted by the genetic component or a portion thereof

2. The method according to claim 1, wherein the plant of the Triticum genus is from the species selected from the group consisting of Triticum aestivum, Triticum durum and Triticum spelta.

3. The method according to claim 1, wherein the explant comprises embryonal tissue.

4. The method according to claim 3, wherein the embryonal tissue comprises part of an immature embryo or a mature seed.

5. The method according to claim 1, wherein the property imparted by the genetic component or portion thereof is herbicide resistance or antibiotic resistance.

6. The method according to claim 1, wherein the method has a transformation efficiency of at least 5%.

7. The method according to claim 1, wherein the method has a transformation efficiency comparable to the transformation efficiency of a corresponding method involving selection of a transformed cell based on a property imparted by the genetic component or a portion thereof.

8. The method according to claim 1, wherein the method comprises a treatment to increase a transformation efficiency.

9. The method according to claim 8, wherein the treatment to increase the transformation efficiency results in a transformation efficiency of at least 5%.

10. The method according to claim 8, wherein the treatment to increase the transformation efficiency comprises at least one treatment selected from the group consisting of:
i. physical and/or chemical damage to the explant or a portion thereof during co-culturing and/or after co-culturing,
ii. centrifugation of the explant before co-culturing and/or during co-culturing and/or after co-culturing,
iii. addition of silver nitrate and/or copper sulfate to a co-culturing medium,
iv. thermal treatment of the explant before co-culturing and/or during co-culturing,
v. pressure treatment of the explant before co-culturing and/or during co-culturing and/or after co-culturing,
vi. co-culturing of the explant with at least one bacterial cell from the Rhizobiaceae family in the presence of a powder and
vii. addition of cysteine to a co-culturing medium.

11. The method according to claim 1, wherein step (b) yields non-chimeric transgenic plants with an incidence of at least 15%.

12. The method according to claim 1, wherein the method further comprises the step of:
(c) selecting the regenerated transgenic plant produced in step (b).

13. The method according to claim 12, wherein the selection in step (c) is based on the detection of the genetic component or a portion thereof.

14. A transgenic plant of the Triticum genus which was produced by the method according to claim 1 or a progeny, or a portion thereof, or a seed thereof.

15. The method according to claim 3, wherein the embryonal tissue is selected from the group consisting of radicula, embryonic axis, scutellum, and nucleus.

16. The method of claim 1, wherein the genetic component is a nucleic acid molecule.

17. The method of claim 1, wherein the at least one bacterial cell from the Rhizobiaceae family is a bacterial cell from the Agrobacterium tumefaciens species.
